(19) United States
(12) Patent Application Publication
(10) Pub. No.: US 2025/0235327 A1
(43) Pub. Date: Jul. 24, 2025

(54) TOOLS AND METHODS FOR ARTHROSCOPIC SURGERY

(71) Applicant: MD-S LLC, Boulder, CO (US)

(72) Inventors: Jacob Segil, Boulder, CO (US); Omer Mei-Dan, Boulder, CO (US)

(21) Appl. No.: 19/175,968

(22) Filed: Apr. 10, 2025

Related U.S. Application Data
(63) Continuation of application No. 18/251,993, filed on May 5, 2023.
(60) Provisional application No. 63/110,083, filed on Nov. 5, 2020.

(51) Int. Cl. A61F 2/46; A61B 17/00; A61B 17/32; A61B 17/56; A61F 2/28

(52) U.S. Cl. CPC A61F 2/4601; A61B 17/320016; A61B 2017/00424; A61B 2017/320008; A61B 2017/564; A61F 2002/2835

(57) ABSTRACT

Tools and methods for use in arthroscopic bone grafting procedures, for example. A bone grafting tool can include a cannulated shaft having a first and second sections, where the first and second section may define a flow path between a first end and a second end of the shaft. The bone grafting tool can include a shaped head coupled to the second section at the second end of the shaft. The head can include an aperture in flow communication with the second section, where the aperture may be positioned at or near normal to an axis of the second section. The second section can be coupled to the first section at an angle, and the first end of the shaft can include means for attachment to a syringe containing bone grafting material to be used in the disclosed methods.

### CROSS-REFERENCE TO RELATED APPLICATIONS

This application is a continuation patent application of U.S. patent application Ser. No. 18/251,993, filed May 5, 2023, and titled “Tools and Methods for Arthroscopic Surgery,” which is a National Stage application of International Application No. PCT/US2021/058155, filed Nov. 5, 2021, and titled “Tools and Methods for Arthroscopic Surgery,” and claims the benefit of U.S. Provisional Patent Application No. 63/110,083, Nov. 5, 2020, and titled “Tools and Methods for Arthroscopic Surgery,” the disclosures of which are incorporated herein by reference in their entirety.

### TECHNICAL FIELD

Various embodiments of the present technology generally relate to surgical tools. More specifically, some embodiments of the present technology relate tools and methods for arthroscopic surgery.

### BACKGROUND

Arthroscopic surgery may include multiple steps and procedures including, for instance, repair of torn cartilage, resection of excess bone, soft tissue removal, shaving of pathological soft tissue, and bone grafting. Many of these procedures are relatively new and for some joints and applications, there is no designated tool or set of tools to complete the procedure efficiently or safely. Some existing medical devices used in arthroscopic surgery during the aforementioned steps are designed in a one size fits all fashion and are not adaptable for use in varying pathological morphologies or different anatomical environments in different joints. As such, known arthroscopic surgery methods may not be possible in certain joints due to the size, shape, trajectory of operating (“working”) portals, and features of existing arthroscopic tools.

Known bone graft delivery devices and related tools like shavers and tissue removal tools may be designed specifically for a specific joint or anatomy, shapes, relative positions and other characteristics. Surgeons may select specific tools to meet specific patients' needs. Accordingly, using standard of care methods with such known tools and devices, surgeons may need to special order or custom manufacture the set of specific tools they require to successfully perform the respective procedure on a specific patient. It is important to select the best suited tools to ensure safe surgical procedure and successful outcomes for patients being treated, and minimize mistakes or undue difficulty during the procedures, along with any undesirable side effects. For these and other reasons the added costs and time required to obtain, learn to use, prepare and deploy the best suited tools for particular patient procedures is justified, but such costs may be passed along to patients.

Accordingly, a need exists for technology that overcomes the problem demonstrated above, as well as one that provides additional benefits. The examples provided herein of some prior or related systems and their associated limitations are intended to be illustrative and not exclusive. Other limitations of existing or prior systems will become apparent to those of skill in the art upon reading the following Detailed Description.

### DRAWINGS

Embodiments of the present technology will be described and explained through the use of the accompanying drawings.

FIG. 1 depicts a perspective view of an example bone grafting tool and associated surgical tool kit.

FIGS. 2A and 2B depict perspective and side views, respectively, of an example second section and attached distal head that can be used with the bone grafting tool and surgical tool kit shown in FIG. 1.

FIG. 3 depicts a perspective view of an example shaft and attached head that can be used with the bone grafting tool and surgical tool kit shown in FIG. 1.

FIG. 4 depicts a perspective view of an example handle that can be used with the bone grafting tool and surgical tool kit shown in FIG. 1.

FIG. 5 depicts a perspective view of an example shaver tool having an attached shaver accessory that can be used with the surgical tool kit shown in FIG. 1.

FIG. 6 depicts a perspective view of an example shaver accessory that can be used with the shaver tool shown in FIG. 5.

FIGS. 7A and 7B depict perspective views of example soft tissue removal tools that can be used with the surgical tool kit shown in FIG. 1.

FIGS. 8A and 8B depict perspective and side views, respectively, of a femoral head microfracture pick that can be used with the surgical tool kit shown in FIG. 1.

FIGS. 9A-9E depict perspective and side views of example distal segments that may be used with the soft tissue removal tools or microfracture picks shown in FIGS. 7A, 7B, 8A and 8B.

FIG. 10 depicts a flow chart of an example method of using a bone grafting tool or surgical tool kit in a patient in need of treatment for a bone abnormality.

FIG. 11 depicts a flow chart of several embodiments of the method shown in FIG. 10.

FIG. 12 depicts another perspective view of the bone grafting tool shown in FIG. 1.

FIG. 13 depicts another perspective view of the bone grafting tool shown in FIG. 1.

FIG. 14 depicts a perspective view of another example bone grafting tool.

FIG. 15 depicts a perspective view of yet another example bone grafting tool with the opposite orientation of the distal head to the shaft of the tool.

FIG. 16 depicts a perspective view of an example shaft and distal head that can be used with the bone grafting tool and surgical tool kit shown in FIG. 1.

FIG. 17 depicts a perspective view of another example shaft and distal head that can be used with the bone grafting tool and surgical tool kit shown in FIG. 1.

FIG. 18 depicts a perspective view of yet another example shaft and distal head that can be used with the bone grafting tool and surgical tool kit shown in FIG. 1.

FIGS. 19A and 19B depict perspective and side views, respectively, of another example bone grafting tool having a straight shaft and a head aperture aligned with the shaft axis.

FIGS. 20A-20C depict side, pan and perspective views, respectively of an example soft tissue removal tool having a cutting edge that can be used with the surgical tool kit shown in FIG. 1.

The drawings have not necessarily been drawn to scale. Similarly, some components and/or operations may be separated into different blocks or combined into a single block for the purposes of discussion of some of the embodiments of the present technology. Moreover, while the technology is amenable to various modifications and alternative forms, specific embodiments have been shown by way of example in the drawings and are described in detail below. The intention, however, is not to limit the technology to the particular embodiments described. On the contrary, the technology is intended to cover all modifications, equivalents, and alternatives falling within the scope of the technology as defined by the appended claims.

### SUMMARY

Various embodiments of the present technology generally relate to devices, surgical tool kits, and methods for arthroscopic cartilage repair and bone grafting. More specifically and particularly, some embodiments relate to devices, surgical tool kits, and methods for use in surgical procedures targeting subchondral cysts in patients' hips, knees, or shoulders.

The disclosed medical devices and associated methods of use include features and steps that address one or more of the drawbacks of know tools and methods for arthroscopic surgery. In some embodiments, the tools according to the present technology are designed modularly such that their dimensions, shapes and other functional and structural characteristics can be tailored to enable surgeons to assemble a tool for a specific purpose for a particular patient and joint's anatomical and pathological characteristics. The pieces can be included in a surgical tool kit. Among the technical and practical benefits of the disclosed embodiments are surgeons' ability to train to use a single modular tool or set of related tools, flexibility and ease of use for most any case that may be encountered in the fields of cartilage repair and bone grafting arthroscopic surgery and, in particular, as applied to hip, knee or shoulder joint-related procedures, and design configurations that are highly effective in carrying out the mechanical effects during operations, while minimizing risk of mistakes and undesirable side effects in patients.

A first aspect of the disclosure provides a bone grafting tool. The bone grafting tool may include a rigid cannulated shaft including a first section and a second section coupled to the first section. The first and second sections may define a flow path between a first end and a second end of the shaft. The bone grafting tool may also include a distal head coupled to the second section at the second end of the shaft. The head may include an aperture in flow communication with at least the second end of the shaft and, in some examples, with the first section as well. In one embodiment of the first aspect, the aperture is positioned at or near normal to an axis of the second section. In another embodiment of the first aspect, the head is at least partially: spherical, spheroid, ovoid, or ellipsoid, shaped, either instead of, or in addition to, the aperture being positioned at or near normal to the axis of the second section. The bone grafting tool allows, for example and without limitation, bone graft material to be delivered into sub-chondral cysts inside the hip joint using standard of care methods. The head provides a bulbous conduit for delivery of bone graft material to any location within a hip joint accommodating and overcoming the unique anatomical concavity. Features of the bone grafting tool according to the present technology enable precise deployment of bone graft material by surgeons and prevention of bone graft material from being released into the joint. The bone grafting tool may include an ergonomic handle for ease of use by a surgical team.

A second aspect of the present disclosure provides a tissue shaver accessory. The shaver accessory may be coupled to a working end of a shaver tool. The shaver accessory may include a cavity having an opening which, when the shaver accessory is coupled to the working end of the shaver tool, is in flow communication with an internal cavity of the shaver tool with the opening of the cavity positioned at or near normal to an axis of the working end of the shaver tool. The shaver accessory according to the present technology may attach to known shavers to block cyst cavity re-opening (where bone graft material was placed in) while shaver is operated with suction that creates a local vacuum environment. This will secure the bone graft material which was placed in the cystic cavity while removing excess material/tissue from a joint such as a hip joint. The shaver accessory may include a shaped crown to cover a cyst opening while the shaver sucks out the excess tissue on the other side, while protecting sensitive tissues/bone graft material in the hip joint while removing excess material. A threaded fastening mechanism allows for use with existing shavers and ensure permanent fixturing.

A third aspect of the present disclosure provides a soft tissue removal tool. The soft tissue removal tool may include a rigid shaft having a bent or curved distal segment terminating in a pointed, or barbed, tip. The soft tissue removal tool according to the present technology may be sized and dimensioned to facilitate maneuvering around the head of the femur to remove torn cartilage and place micro-fracture(s) in the medial femoral head, for example. At least some known tools for this purpose may be straight or less angled and are missing the shaft curvature to accommodate the femoral head convexity and are thus unable to access the most medial part of the femoral head to perform these procedures. The disclosed soft tissue removal tool may include pointed or conical tips that can be positioned at multiple angles in order to provide appropriate orientation with respect to the cystic cavity so as to enable efficient removal of the cyst soft tissue liner prior to grafting. The various tip geometries according to the disclosed embodiments thus enable removal of soft tissue as needed from cystic cavities of joints such as the hip joint, including from different areas thereof. A threaded shaft may allow for the tips to be replaced throughout a surgery using a single handle that may have ergonomic features to facilitate large forces to be transferred to the soft tissue for removal.

A fourth aspect of the present disclosure provides a surgical tool kit. The kit may include several bone grafting tool pieces to enable, for example and without limitation, surgeons to construct a bone grafting tool sized and shaped, and otherwise configured, in a manner best suited to the particular needs of a patient undergoing arthroscopic surgery. For example, the kit may include shaft pieces, distal heads and handles that may be assembled to suit the particular needs of a surgeon. The kit according to the present technology may also include at least one shaver accessory to be deployed with existing and commercially available shaver tools, as needed for a patient at hand. For instance, the kit may include two or more shaver accessories having dimensions, shapes, and aperture or opening sizes that fit the needs of the same or different patient(s) undergoing arthroscopic surgery. The disclosed surgical tool kit may further include several soft tissue removal tool pieces to enable surgeons to fashion a soft tissue removal tool sized and shaped, and otherwise configured, in manner best suited to a patient's particular needs. In an example, the kit may include two or more tipped shaft pieces or two or more handles having dimensions, shapes, angles or arc radii that allow surgeons to tailor the tool to the needs of a particular patient being operated on.

In the fourth aspect of the present disclosure, the surgical tool kit may include a first shaft section, and a second shaft section. The surgical tool kit may include a head for coupling to the second section at a second end of the shaft. In the embodiment of the fourth aspect, the first and second shaft sections, when coupled together, may form a rigid cannulated shaft defining a flow path between a first end and the second end of the shaft. Also, in the embodiment of the fourth aspect, the head includes an aperture and, when the head is coupled to the second end of the shaft, the aperture is in flow communication with the at least the second section and, in some examples, with the first section as well. In one embodiment of the fourth aspect, the aperture may be positioned at or near normal to an axis of the second section of the shaft. In another embodiment of the fourth aspect, the head is at least partially: spherical, spheroid, ovoid, or ellipsoid, shaped, either instead of, or in addition to, the aperture being positioned at or near normal to the axis of the second section.

A fifth aspect of the present disclosure provides a bone grafting tool. The bone grafting tool may include a rigid cannulated shaft defining a flow path between a first end, and a second end, of the shaft. The bone grafting tool may also include a bulbous head coupled to the second end of the shaft. The bulbous head may include an aperture in flow communication with the flow path of the shaft. In one embodiment of the fifth aspect, the aperture may be positioned at or near normal to an axis of at least the second section.

A sixth aspect of the present disclosure provides a surgical tool kit. The surgical tool kit may include a first shaft section, and a second shaft section for coupling to the first section. The surgical tool kit may also include a bulbous head for coupling to the second section at a second end of the shaft. When coupled together, the first and second shaft sections may form a rigid cannulated shaft defining a flow path between a first end and the second end of the shaft. The bulbous head may include an aperture which, when the bulbous head is coupled to the second end of the shaft, is in flow communication with the first and second sections of the shaft. In one embodiment of the sixth aspect, the aperture may be positioned at or near normal to an axis of at least the second section.

A seventh aspect of the present disclosure provides a surgical tool. The surgical tool may include a rigid cannulated shaft including a first section, and a second section coupled, or couplable, to the first section. The first and second sections may define a flow path between a first end and a second end of the shaft. The surgical tool may include a shaver accessory coupled to the second section at the second end of the shaft. The shaver accessory may include an internal cavity having an aperture in flow communication with the second end of the shaft. In one embodiment of the seventh aspect, the aperture may be positioned at or near normal to an axis of the second section. In another embodiment of the seventh aspect, at least a portion of the shaver accessory may be at least partially: spherical, spheroid, ovoid, or ellipsoid, shaped, either instead of, or in addition to, the aperture being positioned at or near normal to the axis of the second section.

An eighth aspect of the present disclosure provides a surgical tool. The surgical tool may include a first rigid shaft, and a second rigid shaft coupled, or couplable, to an end of the first rigid shaft. A first section of the second rigid shaft may include a cutting edge formed in, or on, at least a portion of the first section. The cutting edge may be formed in, or on, the at least a portion of the first section opposite the end of the first rigid shaft. In one embodiment of the eighth aspect, the first rigid shaft, and the second rigid shaft, of the surgical tool may be formed as a unitary construction into a single shaft piece. In another embodiment of the eighth aspect, at least one of the first, and second, rigid shaft(s) of the surgical tool may include one or more bends formed therein, either instead of, or in addition to, the first, and second, rigid shafts being formed as a unitary construction into a single shaft piece.

A ninth aspect of the present disclosure provides a method of using the bone graft tool of, for example and without limitation, the first aspect of the disclosure. The method may include the step of introducing a bone graft material into an interior of the rigid cannulated shaft of the bone graft tool proximate to the first end of the shaft. The method may also include the step of first positioning the second end of the rigid cannulated shaft to, or proximate to, a target site of the bone abnormality. The method may further include the step of depositing the bone graft material to the target site through the aperture or other opening at the second end of the shaft. In some embodiments, the method according to the present technology further provides steps for using the shaver accessory and/or the soft tissue removal tool of, for example and without limitation, embodiments described herein. In some example, a surgeon may deploy those tools in addition to, or instead of, the bone grafting and/or other tools of, for example and without limitation, embodiments described herein.

### DETAILED DESCRIPTION

Reference in this specification to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but no other embodiments.

As used herein, the words “about,” “near,” or “substantially” used in connection with a shape, physical property, or measurable value such as a length, angle, or other dimension means that the stated value may vary within a range of values. In some embodiments, the range of values may be defined by a tolerance, precision, or accuracy of a measurement device used to set or measure the value. In another embodiment, the range of values may be defined as the stated value ±25%. In yet another embodiment, the range of values may be defined as the stated value ±20%. In still another embodiment, the range of values may be defined as the stated value ±15%. In another embodiment, the range of values may be defined as the stated value ±10%. In yet another embodiment, the range of values may be defined as the stated value ±5%. In still another embodiment, the range of values may be defined as the stated value ±2.5%. In another embodiment, the range of values may be defined as the stated value ±1%. In yet another embodiment, the range of values may be defined as the stated value ±a tolerance set by an identifiable art recognized standard, as may be provided by standards setting organizations such as American National Standards Institute (ANSI), National Institutes of Standards and Technology (NIST), and the like. In still another embodiment, the range of values may be defined as the stated value ± and/or − a range of values that a person having ordinary skill in the art would find acceptable for the described application using acceptable materials of construction and fabrication methods, for example.

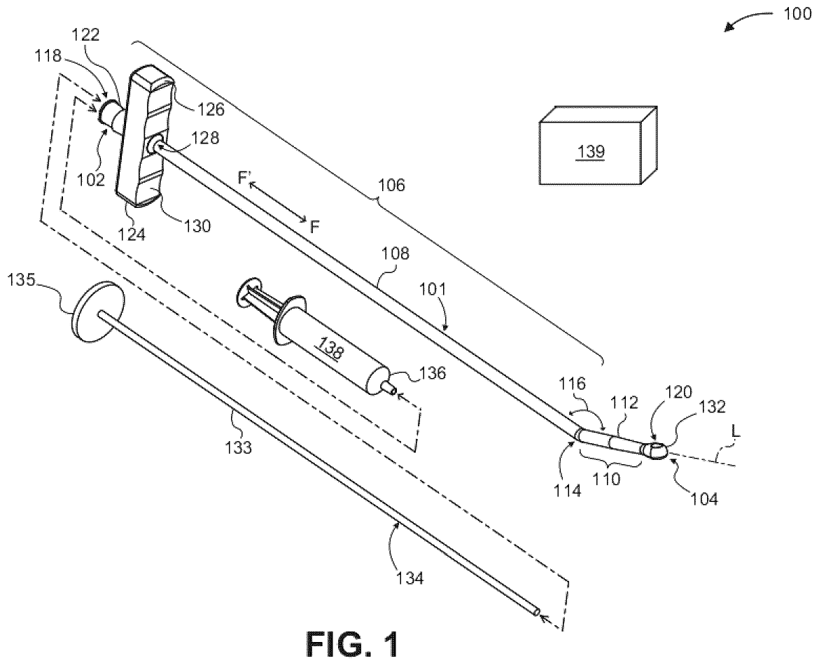

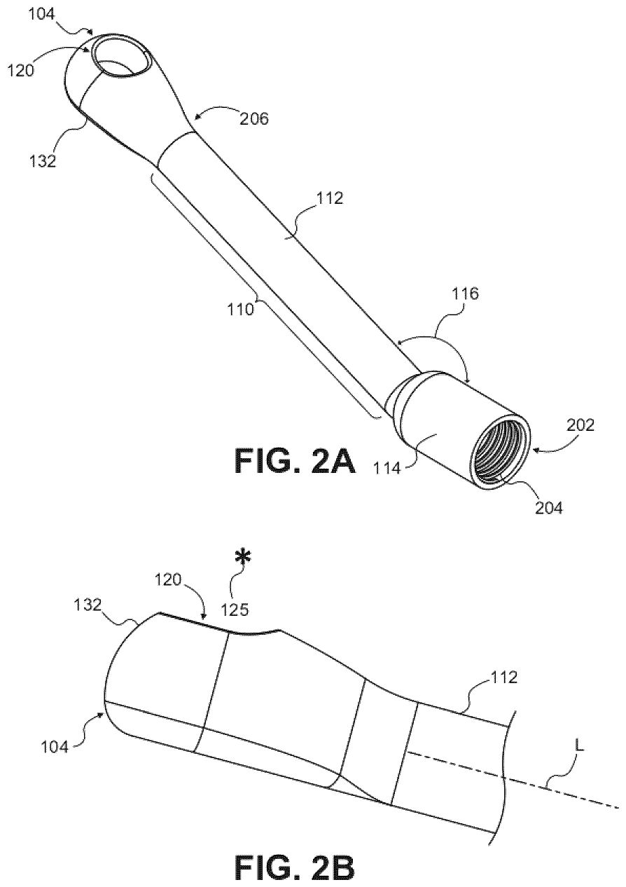

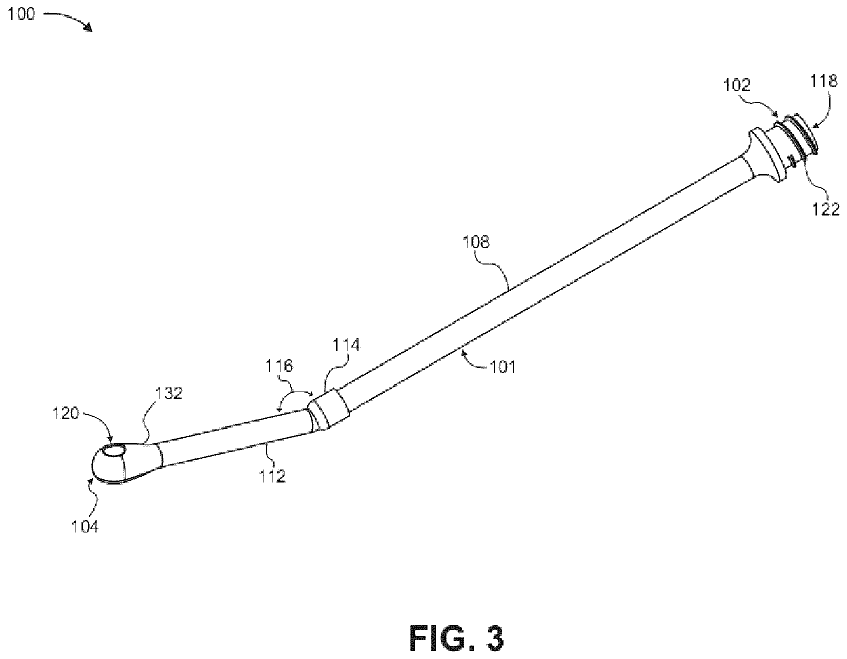

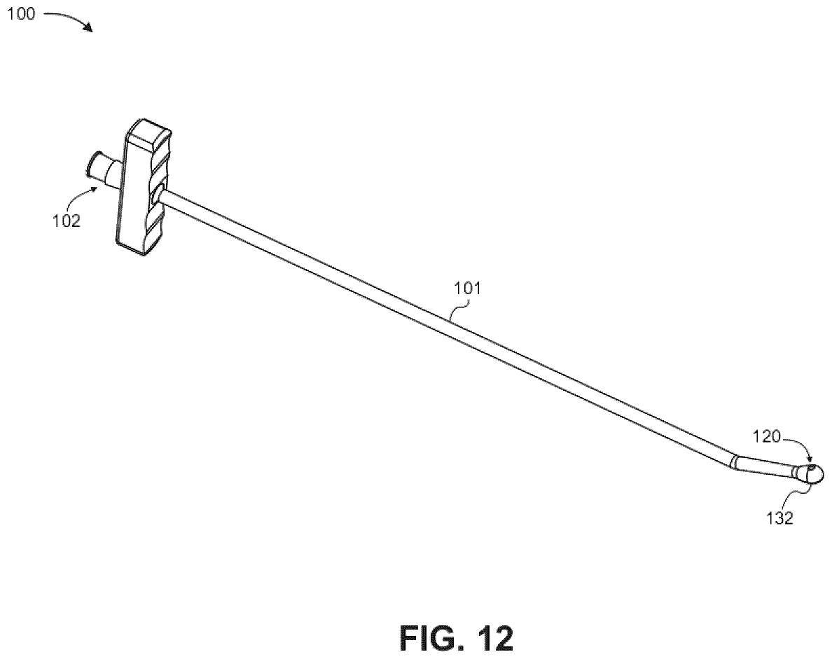

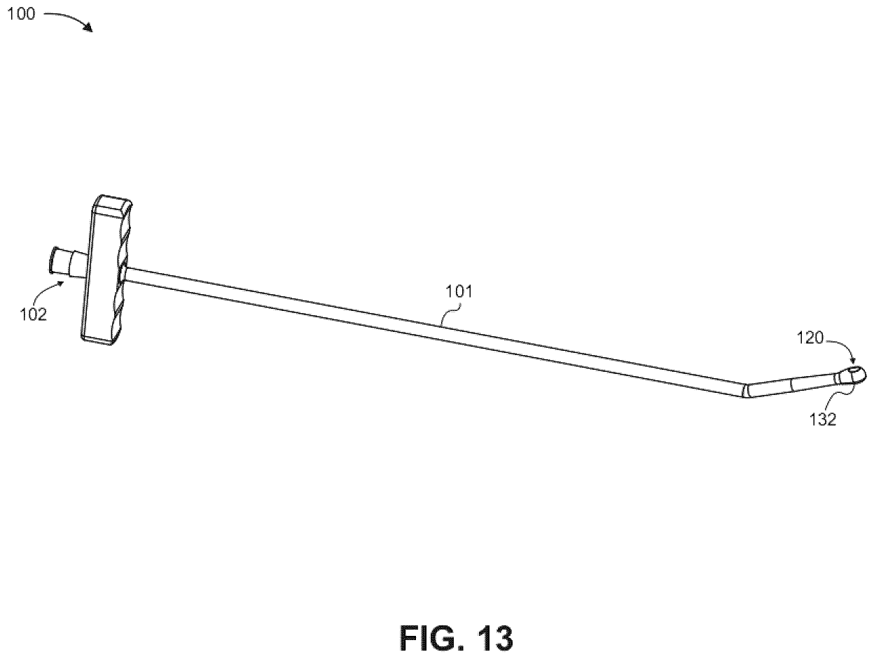

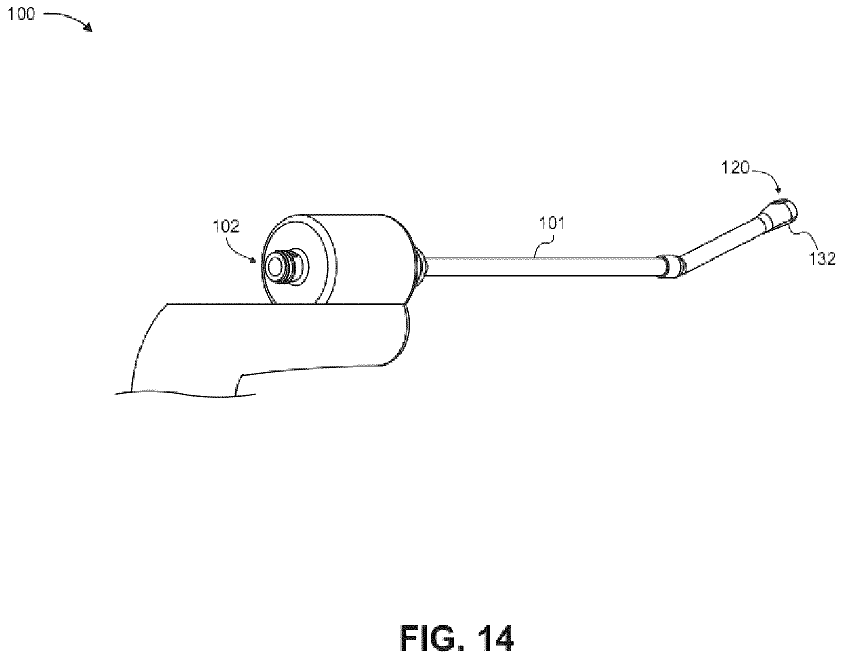

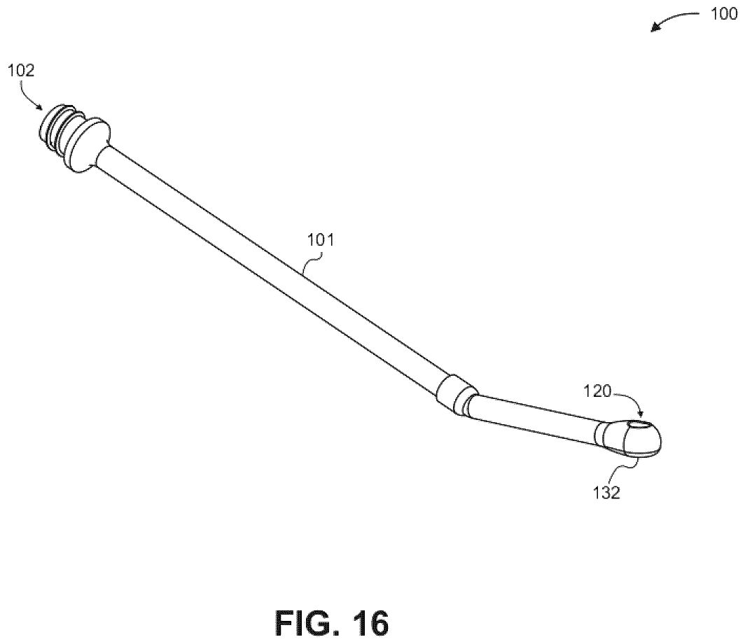

FIG. 1 depicts a perspective view of a bone grafting tool (100) according to an example of the present technology. In one embodiment, bone grafting tool (100) may be a part of a surgical tool kit. Bone grafting tool (100)—hereinafter referred to more succinctly as “tool” (100)—includes a rigid cannulated shaft (101), or simply “shaft” (101). In an example, shaft (101) and possibly also other components of tool (100) described herein, is/are formed of a material such as a metal that is capable of withstanding multiple steam or baking sterilization cycles without deformation or other undesired effects (e.g., stainless steel). In another example, all or part of tool (100) including shaft (101) is formed of a material like a plastic or like polymer suitable for one time (e.g., irradiation) sterilization and packaging, but intended for use in a single-use, disposable tool (100) embodiments. Shaft (101) includes a first section (106) and a second section (110) coupled, or couplable, to the first section (106). When coupled together, first section (106) and second section (110) section define a flow path (F, F′) between a first end (102) and a second end (104) of the shaft (101). In some embodiments, as shown in FIGS. 1, 12, 13 and 16-18, for example, shaft (101) is continuously formed with first (106) and second (110) sections as a one-piece construction. In other embodiments, as illustrated in FIGS. 2A, 3, 14 and 15, for example, second section (110) may be detachably coupled to the first section (106) by way of a means (114) for detachably coupling the two shaft (101) sections (106, 110) together. For instance, such means (114) may include mating screw threads for the detachable coupling, as shown in FIG. 2A.

In the embodiment shown in FIG. 1, a length of first section (106) may be greater than a length of second section (110). In the embodiment shown in FIG. 1, an overall length of tool (100) between first (102) and second (104) ends may be about (e.g., ±10%) 16 cm. In other examples, the overall length of tool (100) between first (102) and second (104) ends may be from about 8 cm to about 24 cm. In yet other examples, the overall length of tool (100) between the first (102) and second (104) ends may be from about 12 cm to about 20 cm. The overall length of tool (100) is dictated by the respective lengths of the first (106) and second (110) sections, and in some embodiments, also by trigonometry.

In the embodiment shown in FIG. 1, the second section (110) can be coupled to the first section (106) at an angle (116). In an example, angle (116) may be about (e.g., ±10%) 15° (degrees). In other examples, angle (116) is about 5°, or about 10°, or about 20°, or about 25°. In yet other examples, angle (116) is about 7.5°, or about 12.5°, or about 17.5°, or about 22.5°. When embodied as, or included in, a surgical tool kit—hereinafter recently to more simply as “kit”—shaft (101) may be embodied as a multiple (e.g., two) piece shaft (101) with one or more lengths of first section(s) (106) detachably couplable to one or more lengths of second section(s) (110) included in the kit. Thus, a user of the kit can fashion a tool (100) having a first pair of desired section (106, 110) lengths (and thus a first desired overall tool (100) length) for use on a first patient having a first set of particular anatomical sizes and arrangements in, for example, hip, shoulder or knee bone grafting procedures. For a second patient in a similar type of bone grafting procedure, the user of the kit can assemble the tool (100) having a second set of desired lengths to suit the second patient's unique anatomical sizes and arrangements. Similarly, kit may contain two or more detachable second sections (110) so as to fashion tool (100) with varying angles (116) to suit the particular needs (e.g., adult vs. pediatric, or human vs. veterinary) of the patient being operated on in, for example, arthroscopic surgery procedures.

Referring to FIGS. 1, 2A, 2B and 3, tool (100) may include a distal head (132) coupled, or couplable, to second section (110) at second end (104) of shaft (101). When so coupled to second section (110), head (132) thereby defines the second end (104). In the illustrated embodiments, head (132) may include an aperture (120) in flow communication with the second section (110), and thus also in flow communication with first section (106). As shown in FIGS. 2A and 2B, aperture (120) may be positioned at or near (e.g., ±10%) normal to a longitudinal axis (L) of the second section (110). In some embodiments, as shown in FIG. 2B for instance, head (132) is flushly coupled to the end of a head shaft (112) of second section (110), but extends therefrom asymmetrically in at least one of three dimensions with respect to the L axis. In the example shown in FIG. 2B, the material of construction of head (132) bulges toward a first radial side of head shaft (112) such that aperture (120) is displaced away from axis L. In some embodiments, as illustrated in the figures, the first radial side is the side facing generally in the same direction as angle (116).

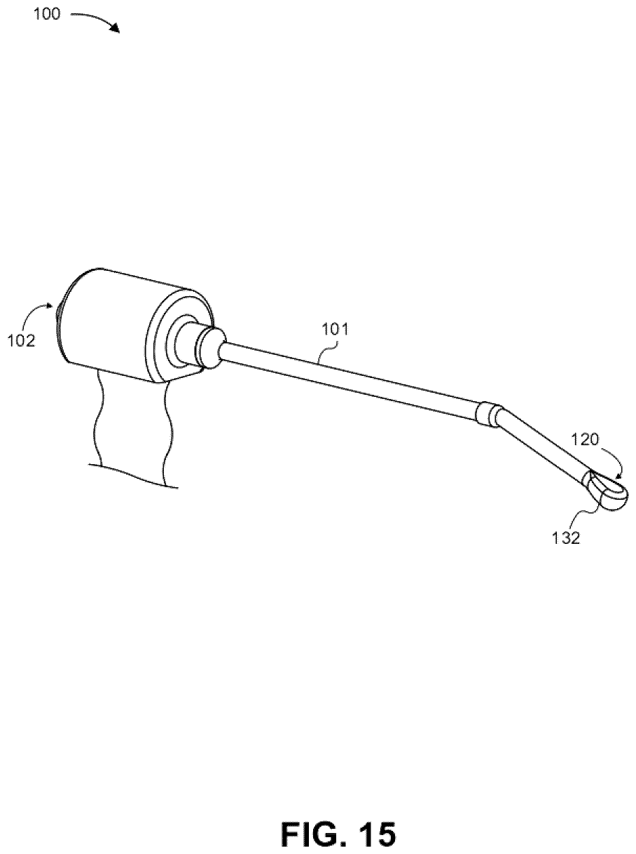

In some embodiments, not shown in the figures, head (132) may be detachably coupled to second section (110) by way of a detachable coupling means (206) (e.g., mating screw threads, press-fit or other suitable means known to persons having ordinary skill in the art). In the examples shown in FIGS. 15-18, head (132) may be formed continuously with the second section (110) and, optionally, also the first section (106). Such fully one piece constructions may be desirable in some applications for ease or effectiveness of use and related operations such as cleaning and sterilization of tool (100) and its components parts.

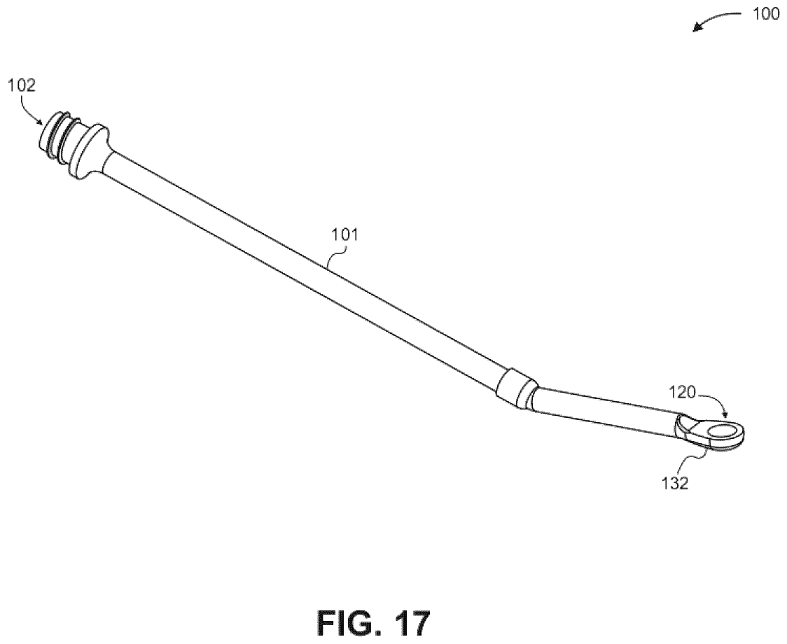

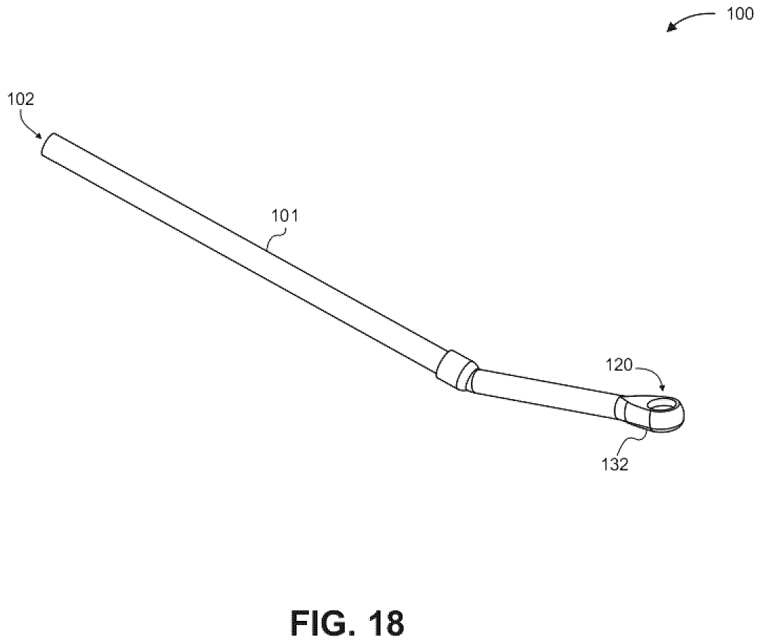

In one embodiment, head (132) may be at least partially spherical in shape. In another embodiment, head (132) may be at least partially spheroid in shape. In yet another embodiment, head (132) may be at least partially ovoid in shape. In still another embodiment, head (132) may be at least partially ellipsoid in shape. Variations on the shapes, dimensions, and positions of head (132) and aperture (120) by persons having ordinary skill in the art are expected, but without departing from the spirit and scope of the present disclosure. Some such variations are shown in FIGS. 2A, 2B and 12-18. For instance, head (132) shown in FIG. 2B is flattened on its side opposite aperture (120), while heads (132) shown in FIGS. 17 and 18 have a flattened side on the same side as aperture (120). Shapes and configurations of head (132), along with one or both of shaft (101) inner diameter and aperture (120) bore diameter, may be design considerations to be taken into account for tool (100) to effect operational characteristics including, without limitation, material (e.g., bone graft material (138)) flow rates through shaft (101) in either the F flow direction or the opposite F′ direction. In some embodiments, head (132) made be formed of metal. In other embodiments, head (132) may be formed of plastic. In an example, a plastic head (132) may be formed of a clear plastic enabling better visualization of its function during surgical use.

Surgical tool kit may include two or more detachable heads (132) having varying size, shape, or aperture (120) size attributes. A user of the kit can thus fashion a tool (100) having a first head (132) with a first desired set of attributes for use on a first patient having a first morphology (e.g., a first chondral cyst size or shape or location) at a target site (125) of a bone abnormality. For a second patient with a second morphology (e.g., a second chondral cyst size or shape or location), the user of the kit can prepare the tool (100) having a second set of desired attributes to suit the second patient's unique morphology. Similarly, kit may contain two or more detachable second sections (110) with differing heads (132) so as to fashion tool (100) having differing attributes to suit the particular needs of different patients being operated on in, for example, arthroscopic surgery procedures.

In one embodiment, a bore (e.g., inner) diameter of first section (106) is about equal to a bore (e.g., inner) diameter of second section (110). In another embodiment, bore diameter of first section (106) is greater than a bore diameter of at least a portion (e.g., proximate to second end (104)) of second section (110). In an example, second section (110) is tapered, having a first bore diameter proximate to first section (106) that is greater than a second bore diameter proximate to second end (104). In another embodiment, tapered second section (110) has first bore proximate to first section (106) that is less than second bore diameter proximate second end (104). Variations in bore diameter of portions of shaft (101) may be design considerations of tool (100) and kit that provide control over differing flow properties of, for instance, a variety of bone graft materials (136) that may be used in methods such as those described herein.

Tool (100), whether it be included, or not included, in the kit, may include a plunger (134) for insertion into an opening (118) at first end (102) of first section (106) of shaft (101), as shown in FIG. 1. Plunger (134) may be formed of a same, similar, or different material as first section (106), and may be either sterilizable or disposable, similarly to as those terms are used in the description above. Plunger (134) includes a rigid plunger shaft (133), and a plunger cap (135) coupled, or couplable, to one end of plunger shaft (133). Plunger shaft (133) is shaped and dimensioned to enable an alternating insertion into, and removal from, first section (106). Thus, for example, for a round and rigid cannulated first section (106), plunger (134) may include a round and rigid plunger shaft (133), as shown in FIG. 1.

A diameter of plunger shaft (133) may be about the same, or less, than bore diameter of first section (106), so as to provide a slip fit ensuring proper function of the plunger in tool (100). In an example, diameter of plunger shaft (133) is about 0.003″ (inches) less than bore diameter of first section (106) proximate to opening (118). In another example, the tolerance of plunger shaft (133) diameter in relation to bore diameter of first section (106) is defined by, and conforms to, ANSI Standard B4.1 R2009. In one embodiment, first section (106) is tapered, having a first bore diameter proximate to opening (118) that is greater than a second bore diameter at the opposite end. In the embodiment, plunger shaft (133) may be similarly and correspondingly tapered so as to provide an effective seal between respective interior surfaces. In any event, plunger shaft (134) is sized and dimensioned to facilitate operation of plunger (134) in the disclosed methods to cause all or a majority of material (e.g., bone graft material (136)) introduced into an interior of first section (106) to be flowed through tool (100) in the direction of flow path F.

Tool (100) may include a means (122) for attaching first end (102) of shaft (101) to a syringe (136). In one embodiment, means (122) for attaching first end (102) of shaft (101) is a Luer lock design configured to removably couple a corresponding mating Luer lock mechanism of syringe (136) to first end (102). When so attached, a Luer fitting results between first end (102) and a nozzle end of syringe (136). In other embodiments, means (122) for attaching first end (102) of shaft (101) to syringe (136) may be screw threads or other suitable designs for alternately attaching, and removing, syringe (136) with correspondingly mating attachment means to and from first end (102), as shown in FIG. 1. In some embodiments, surgical tool kit may include syringe (136) that is pre-filled with bone graft material (138) and, as required, a temperature regulation means (139) (e.g., a cooler) to maintain material (138) at or near a pre-determined temperature or within a range of predetermined temperatures, as dictated, for instance, by a material (138) manufacturer's instructions.

Referring now to FIGS. 1, 4 and 12-14, tool (100), whether it be included, or not included, in the kit, may include a handle (124) for facilitating holding and manipulating tool (100) during use in the disclosed methods. Handle (124) may be formed of a disposable or sterilizable, or otherwise easily and effectively cleanable, material of construction, as described above. In one embodiment, handle (124) is coupled, or couplable, to a portion of shaft (101) (e.g., to first section (104) proximate to first end (104)).

Handle (124) may be formed with various shaped features to provide an ergonomically favorable grip while carrying, holding, applying forces to, and otherwise manipulating tool (100). For example, such ergonomic features may include rounded edges (126) and a fluted section (128) defining boundaries of portions of handle (124). The example handle (124) illustrated in FIGS. 1, 12 and 13 is a T-style handle with an axially centered bore for receiving, and coupling, to shaft (101).

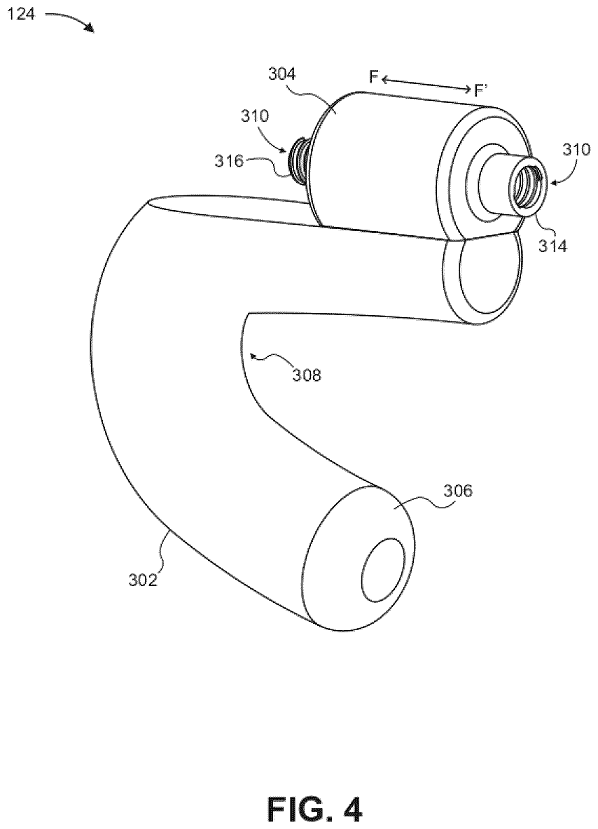

The example handle (124) illustrated in FIGS. 4, 14 and 15 is a grip-style handle with ergonomically-formed features (e.g., 302, 306 and 308). In the embodiment, grip-style handle (124) may include a bore hole (310) formed through a portion of its material of construction (e.g., a disposable, sterilizable material or otherwise effectively and easily cleanable material, as described above) at a top portion (304), but rather than for receiving first section (106) therethrough, first section (106) may instead by detachably coupled, or couplable, to a means (314) (e.g., screw threads or Luer fitting) for attaching first end (104) to a first side of bore (310). On a second side (316) of bore (310) is positioned a means (316) (e.g., screw threads or Luer fitting) for attaching syringe (136), similar to as described above with respect to means (118). This design configuration with bore (310) axially aligned with first section (106) facilitates flow of material (e.g., bone graft material (138)) in the same path (F) as through shaft (101) during operation of tool (100) in the disclosed methods.

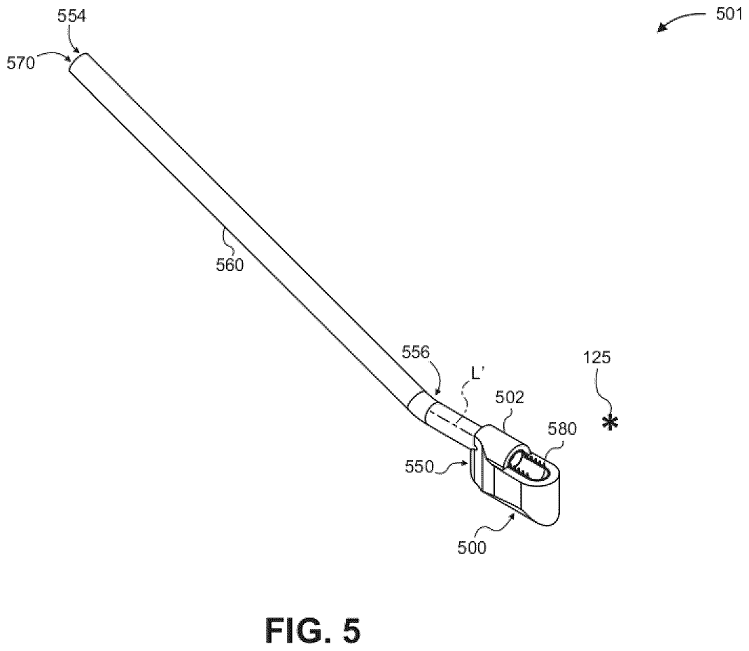

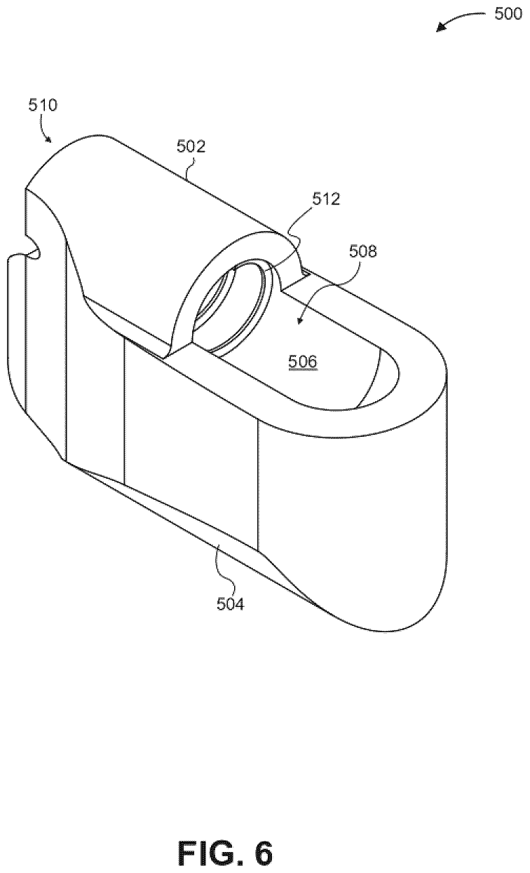

Referring now to FIGS. 5 and 6, in an embodiment, the surgical tool kit according to the present technology may include a shaver accessory (500) coupled, or couplable to, a working end (550) of a shaver tool (501). In an example, the shaver tool (501) may be included in the kit along with the shaver accessory (500). Shaver accessory (500) may include a cavity (506) having an opening (508). When coupled to working end (550), opening (508) is in flow communication with an internal (e.g., tubular) cavity (554) of the shaver tool (501).

As shown in FIG. 5, opening (506) of cavity (508) may be positioned at or near (e.g., ±10%) normal to a longitudinal axis (L′) of the working end (550) of shaver tool (501). Shaver accessory (500) includes a bore hole (512) through a top portion (502) of the accessory (500) and in flow communication with working end (550). In some embodiments, as shown in FIG. 5 for instance, shaver accessory (500) is flushly coupled to working end (550) of shaver tool (501), but extends therefrom asymmetrically in at least one of three dimensions with respect to axis L′. In the example shown in FIG. 5, the material of construction of shaver accessory (500) bulges toward a first radial side of working end (550) such that opening (506) is displaced away from axis L′. In some embodiments, as illustrated in FIG. 6, the first radial side is the side facing generally in the same direction as an angle (556) present along a rigid cannulated shaft (560) of shaver tool (501).

In some embodiments, shaver accessory (500) may be detachably coupled to working end (550) of shaver tool (501) by way of a detachable coupling means (510) (e.g., mating screw threads, or other suitable means known to persons having ordinary skill in the art). Alternatively, shaver accessory (500) may be formed continuously with the working end (550) and, optionally, also the entirety of shaft (560). Such fully one piece constructions may be desirable in some applications for ease or effectiveness of use and related operations such as cleaning and sterilization of shaver tool (501) and its components parts.

In one embodiment, shaver accessory (500) may be at least partially spherical in shape. In another embodiment, shaver accessory (500) may be at least partially spheroid in shape. In yet another embodiment, shaver accessory (500) may be at least partially ovoid in shape. In still another embodiment, shaver accessory (500) may be at least partially ellipsoid in shape. Variations on the shapes or dimensions of shaver accessory (500), cavity (508) and opening (506) by persons having ordinary skill in the art are expected, but without departing from the spirit and scope of the present disclosure. For instance, shaver accessory (500) shown in FIGS. 5 and 6 is flattened on the same side as opening (506). Alternatively, or additionally, shaver accessory (500) may have a flattened side on the side opposite opening (506).

Shapes and configurations of shaver accessory (500), along with at least one of shaft (560) interior cavity (554) inner bore diameter, cavity (508) shape, position and volume, and opening (506) shape, position and extent, may be design considerations to be taken into account for shaver tool (501) (e.g., as used with kit) to effect operational characteristics including, without limitation, material (e.g., shaved material flow rates through shaft (560)). For instance, where shaver accessory (500) enables blades (580) of shaver tool (501) to be at least partially exposed to an exterior of accessory (500), as shown in FIG. 5, for removing an undesirable material or residue from a patient's bone abnormality target site (125) under suction at a first end (570) of tool (501), the configuration and use of shaver accessory (500) according to the present technology facilitates flow of the undesired material or residue from accessory (500) toward first end (570).

According to some embodiments, the disclosed surgical tool kit may include two or more detachable shaver accessories (500) having varying size, shape, or cavity (508) or opening (506) size, shape, or volume attributes. A user of the kit can thus fashion a shaver tool (501) having a first shaver accessory (500) with a first desired set of attributes for use on a first patient having a first morphology (e.g., a first chondral cyst size or shape or location) at a bone abnormality target site (125). For a second patient with a second target site morphology (e.g., a second chondral cyst size or shape), the user of the kit can prepare the shaver tool (501) having a second set of desired attributes to suit the second patient's unique morphology.

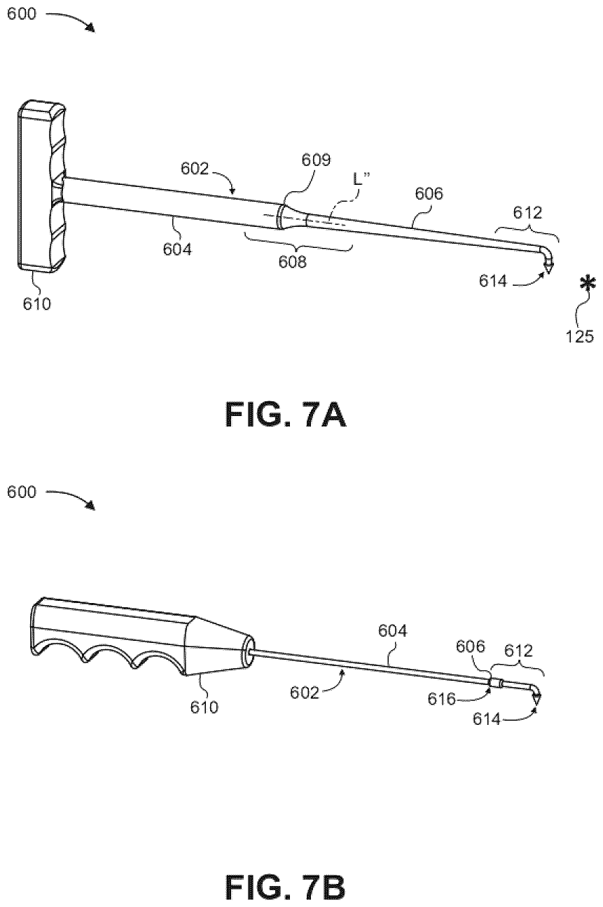

FIGS. 7A and 7B depict perspective views of example soft tissue removal tools (600) that can be used with the surgical tool kit shown in FIG. 1 to, for example, clear cyst or other soft tissue at or near the target site of a bone abnormality. In one embodiment, soft tissue removal tool (600) may be included in the kit shown and described above with reference to FIG. 1. Soft tissue removal tool (600)—hereinafter referred to more succinctly as tool (600)—includes a rigid shaft (602). In an example, shaft (602) may be formed as a solid (e.g., non-cannulated) piece. In another example, shaft (602) may be formed at least in part as a hollow piece so as to reduce a weight of tool (600). In the embodiment shown in FIG. 7A, shaft (602) may be tapered and may have a first section (604) having a first outer diameter and a second section (606) having a second outer diameter that is less than the first outer diameter.

In the illustrated example of FIG. 7A, the first section (604) has a uniform first outer diameter, where a transition section (608) separates the first section (604) from the second section (606) of shaft (602). In the example, the second section (606), instead of the first section (604), may be tapered. In another embodiment, not shown in the figures, shaft (602) may not include transition section (608), and may instead be tapered from first outer diameter to second outer diameter along all or a part of its length. A length of first section (604) may be greater than a length of second section (606). An overall length of tool (600) between each of its ends (including, e.g., an attached handle (610)) may be about 16 cm. In other examples, the overall length of tool (600) may be from about 8 cm to about 24 cm. In yet other examples, the overall length of tool (600) may be from about 12 cm to about 20 cm. The overall length of tool (100) is dictated by the respective lengths of the first (604) and second (606) sections, and may be selected according to the particular anatomical dimensions of, or other practical considerations for, patients upon which tool (600) may be used.

Tool (600) may include a handle (610) coupled, or couplable, to an end of shaft (602) opposite a distal segment (612) of shaft (602). Handle (610) may facilitate holding and manipulating tool (600) during use in the disclosed methods. Handle (610) may be formed of a disposable or sterilizable, or otherwise easily and effectively cleanable, material of construction, as described above. In an example, handle (610) and shaft (602) may be formed as a continuous one-piece construction, which may facilitate effective sterilization procedures for tool (600). In another example, handle (610) may be detachably coupled to shaft (602), as by way of mating screw threads. The embodiment shown in FIG. 7A includes a T-bar style handle (610), while the embodiment shown in FIG. 7B includes a grip style handle (610) axially aligned with shaft (602). Handle (610) may include ergonomic features similar to those described above with respect to handle (124) of bone grafting tool (100). Such ergonomic features as applied to tool (600) handle (610) can facilitate transfer of large forces to the soft tissue for removal.

In embodiments where tool (600) is included in the surgical tool kit described herein, a plurality of handles (610) of varying shapes, sizes and styles may be present to suit the needs of individual users and for specific procedural purposes. For instance, in some embodiments, a T-bar style handle (610) may be sized and shaped to facilitate use of a hammer tool to hit the handle (610) attached to shaft (602) to direct force at or near the target site (125). Similarly, tool (600) embodiments having the tapered shaft (602) may facilitate forceful entry into and through tissues (e.g., through a working portal) during use of tool (600) in surgical procedures.

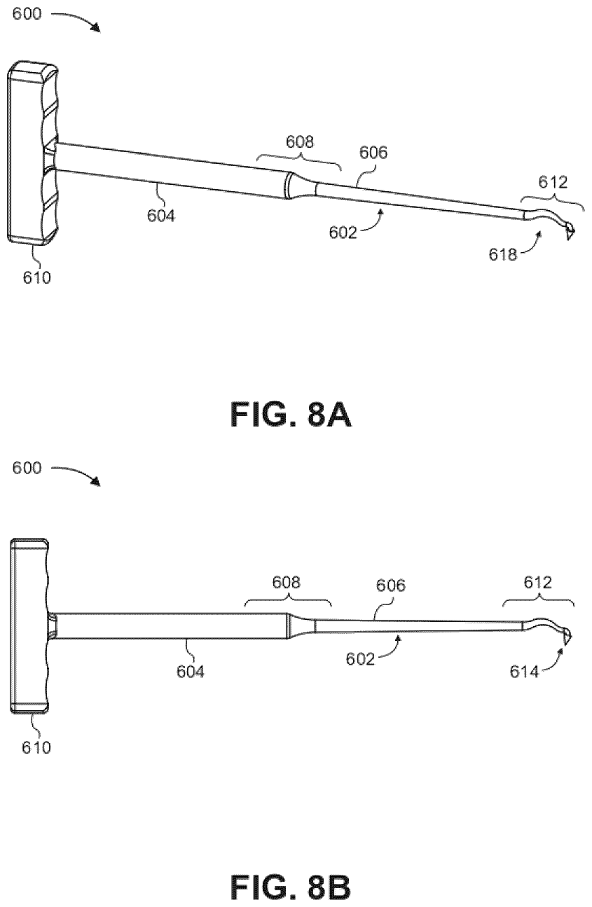

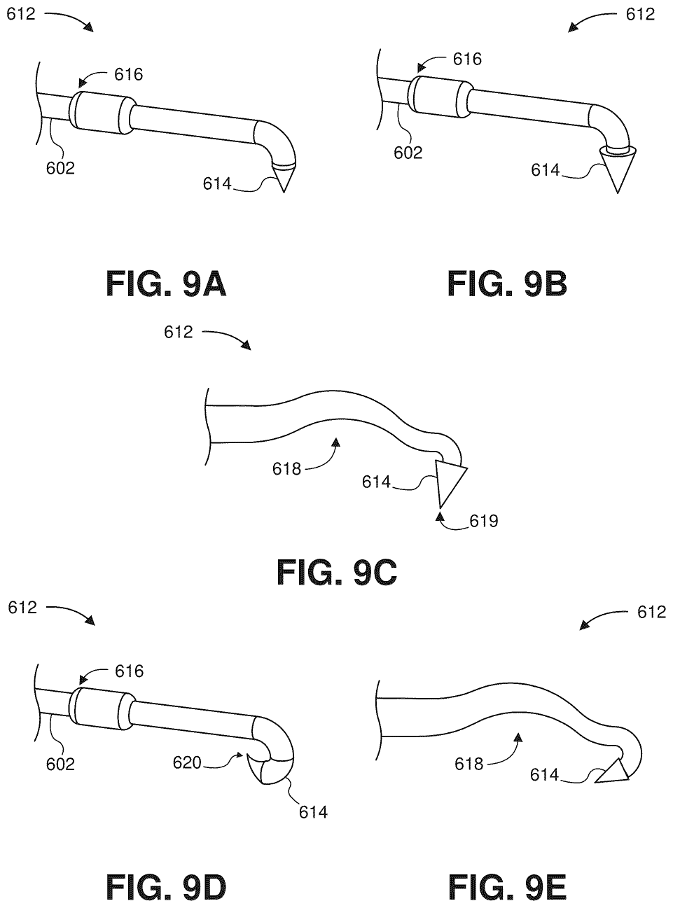

Distal segment (612) of tool (600) may terminate in a tip (614) that is sized and shaped to facilitate capture, and removal, of tissue at or near the target site (125). Tip (614) may be coupled, or couplable, to the end of distal segment (612). In the embodiments illustrated in FIGS. 7A, 7B, 8A, 8B and 9A-9D, tip (614) is conical in shape and has a sharp point (619). A cone base diameter of tip (614) may be greater than (e.g., FIG. 9B) or equal to (e.g., FIG. 9A) an outer diameter of shaft (602) (or second section (606) thereof). The example tip (614) of FIG. 9E takes the shape of a pointed barb (620). In the illustrated embodiments, distal segment (612) may be bent in one or more places, and at one or more angles, relative to a longitudinal axis (L″) of shaft (602). In one embodiment, the bending of distal segment (612) may take the form of a curved bent elbow shape (e.g., FIGS. 7A, 7B, 8A, 8B, 9A, 9B and 9C), where the tip (614) point (619) is oriented with respect to axis (L″) at an angle that is greater than 0° and less than or equal to 90°. In another embodiment, the bending of distal segment (612) may take the form of a curved hook shape (e.g., FIGS. 9D and 9E), where the tip (614) point (619), or barb (620), is oriented with respect to axis (L″) at an angle that is greater than 90°. In other embodiments (e.g., FIGS. 8A, 8B, 9C and 9E), distal segment (612) is formed with more than one bend introduced into it with, for instance, three such bends to form at least one arc (618), with the concave face of arc (618) facing in generally the same direction as tip (614) point (619).

At least some known soft tissue removal tools are straight and devoid of bends or curves. As compared to known embodiments, the above described curved and/or bent distal segments (612) and conical or barbed tips (614) according to the tool (600) of the present technology provide tool (600) users improved abilities to manipulate tool (600) in such a way as to more accurately and effectively maneuver distal segment (612) and tip (614) toward, in, near or on the target site (125). For instance, as employed in knee, shoulder or hip bone grafting procedure, such tool (600) features and various geometries of the distal segment (612) and the tip (614) facilitate maneuvering distal segment (612) and/or tip (614) around the head of the femur to remove (e.g., from hip joint cystic cavities) torn cartilage or other undesired material from at or near the target site (125). In embodiments where tool (600) tips (614) are pointed for use as a microfracture tool, the aforementioned features may facilitate introduction of micro-fracture(s) or perforation(s) in the head of the femur medially. Inclusion of tool (600) in the surgical tool kit according to the present technology may be especially useful in patients presenting with cystic changes with corresponding defects on the femoral head, as is commonly seen in practice scenarios. Known soft tissue removal tools for use in such procedures are straight and thus users thereof are not able to get to the most medial part of the femoral head to perform these procedures. By contrast, the various geometries of the distal segment (612) and tip (614) enables surgeons using tool (600) to remove soft tissue from different areas within the cyst as needed.

In some embodiments, second section (606) may be detachably coupled to first section (604) by way of a detachable coupling means (616) (e.g., mating screw threads, or other suitable means known to persons having ordinary skill in the art). In the examples shown in FIGS. 7A, 8A, 8B, 9C and 9E, shaft (602), or first (604) and second (606) sections of shaft (602) may be formed continuously as a one-piece construction. Such fully one piece constructions may be desirable in some applications for ease or effectiveness of use and related operations such as cleaning and sterilization of tool (600) and its components parts.

When embodied as, or in, the surgical tool kit according to the present technology, shaft (602) may be embodied as a multiple (e.g., two) piece shaft (602) with one or more lengths of first section(s) (604) detachably couplable to one or more lengths of second section(s) (606) included in the kit. As used, for instance, in shoulder, hip or knee bone grafting procedures, a user of the kit can fashion a tool (600) fit for a particularly selected purpose by selecting the forms and numbers of bends or curves of distal segment (612) and/or the angles of orientation at which tip (614) of tool (600) is oriented with respect to axis (L″) in order to provide appropriate lengths, angles and orientations, as the case may be, to suit the different patients' unique anatomical dimensions and arrangements.

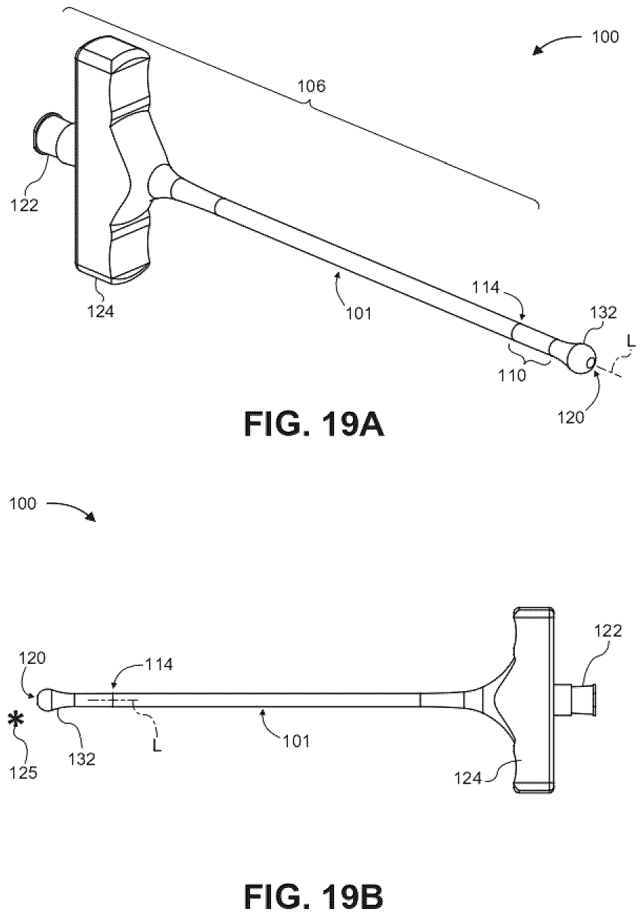

FIGS. 19A and 19B depict perspective and side views, respectively, of another example bone grafting tool (100) having a straight, unbent shaft 101 and a head (132) aperture (120) aligned (e.g., in line with) with the shaft axis (L). The example tool (100) embodiment shown in FIGS. 19A and 19B may be included in the surgical tool kit according to the present technology either in addition to, or instead of, the embodiment of tool (100) shown in FIG. 1. As in the example shown in FIG. 1, tool (100) shown in FIGS. 19A and 19B may include a rigid cannulated shaft (101) defining a flow path between a first end (102) and a second end (104) of the shaft (101). A bulbous head (132) may be coupled to the second end (104) of the shaft (101). As with the embodiments of head (132) as described above with reference to, for example, tool (100) shown in FIG. 1, the head (132) of FIGS. 19A and 19B may include an aperture (120) in flow communication with the flow path of the shaft (101). One difference between head (132) of FIG. 1 and head (132) of FIGS. 19A and 19B is that the aperture (120) of the latter head (132) embodiment may be positioned along an axis (L) of shaft (101).

As with the head (132) embodiments described above with reference to FIG. 1, for example, the bulbous head (132) illustrated in FIGS. 19A and 19B may be detachably coupled to the shaft (101) at the second end (104) thereof. The shaft (101) of the tool (100) shown in FIGS. 19A and 19B may include a first section (106) and a second section (110) detachably coupled to the first section. As compared to the shaft (101) of the tool (100) shown in FIG. 1, second section (110) shown in FIGS. 19A and 19B is not coupled to first section (106) at an angle. Rather, in the tool (100) embodiment shown in FIGS. 19A and 19B, first (106) and second (110) sections of shaft (101) may share a common axis (L). In this embodiment, the bulbous head (132) may be detachably coupled to the second section (110) of the shaft, and first section (106) may be detachably coupled to second section (110). As used in the surgical tool kit according to the present technology, coupling of second section (110) to first section (106) at an angle of about 0°, rather than at an angle (as shown in FIG. 1). Likewise, use of head (132) shown in FIGS. 19A and 19B provides surgical teams greater flexibility in fashioning a bone grafting tool (100) according to a specific patient being operated on and their unique respective anatomy and bone abnormality morphology.

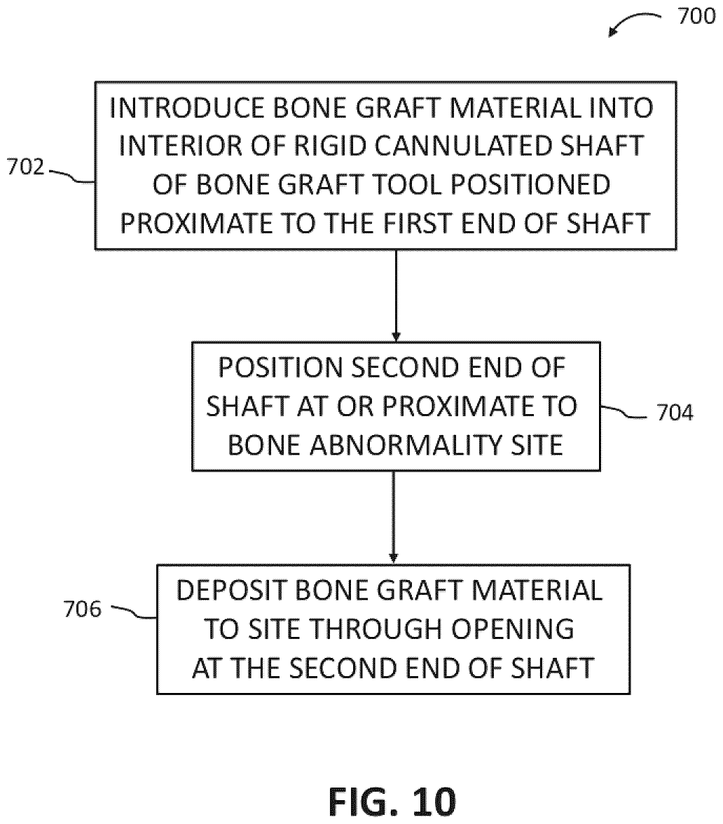

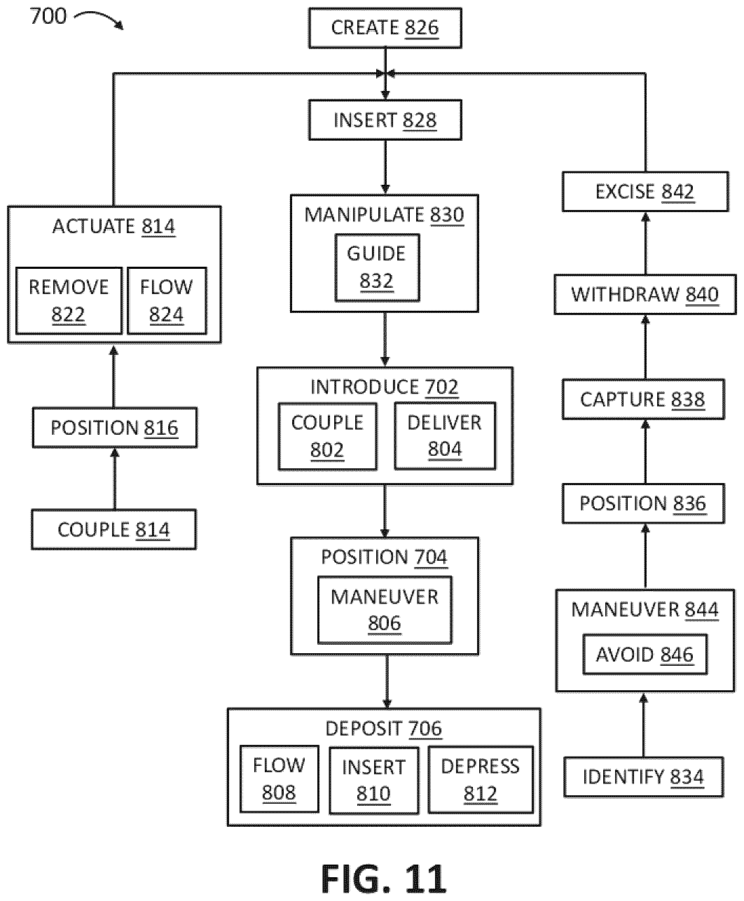

FIGS. 10 and 11 depict flow charts of a method (700) of using a bone graft tool (e.g., tool (100)) or surgical tool kit in a patient (e.g., human or veterinary) in need of treatment for a bone abnormality. Method (700) may, in practice, be performed during an arthroscopic bone grafting procedure, including on a patient's hip, knee or shoulder, or portions thereof (e.g., subchondral cyst(s)). In an embodiment, method (700) may include the step of introducing (702) bone graft material (138) into an interior of the rigid cannulated shaft (101) of the bone graft tool (100) proximate to the first end (102) of the shaft (101). In the embodiment, method (700) may also include the step of first positioning (704) the second end (104) of the shaft (101) at, or proximate to, a target site (125) of the bone abnormality. In the embodiment, method (700) may further include the step of depositing (706) the bone graft material to the target site (125) through the aperture (120) or other opening at the second end (104) of the shaft (101).

In an example of method (700), the step of introducing (702) the bone graft material (138) into the interior of the shaft (101) may include coupling (802) a syringe (136) containing the bone graft material (138) to an opening (118) at the first end (102) of the shaft (101). In this example, the method (700) step of introducing (702) the bone graft material (138) into the interior of the shaft (101) may also include delivering (804) the bone graft material (138) to the interior of the shaft (101) using the syringe (136).

In yet another example of method (700), the step of first positioning (704) the second end (104) of the shaft (101) to, or proximate to, the target site (125) may include maneuvering (806) the head (132) coupled to the second end (104) to, or proximate to, the target site (125). In yet another example of method (700), the step of depositing (706) the bone graft material (138) to the target site (125) may include flowing (808) the bone graft material (138) though the aperture (120) of the head (132).

In still another example of method (700), the step of depositing (706) the bone graft material to the target site (125) may include inserting (810) the plunger (134) into an opening (118) at the first end (102) of the shaft (101). In this example, the step of depositing (706) the bone graft material to the target site (125) may also include depressing (812) the plunger (134) into the interior cavity of the shaft (101) to cause the bone graft material (138) to be flowed (F) through the opening (e.g., head (132) aperture (120)) at the second end of the shaft (101).

In yet another example, method (700) may also include the step of creating (826) a working portal from an exterior of the patient to the target site (125). In this example, method (700) may also include the step of inserting (828) the second end (104) of the shaft (101) into the working portal. In still another example, method (700) may also include the step of manipulating (830) the handle (124) coupled, couplable to, the first section (106) of the shaft (101) to facilitate at least one of: the first positioning (704) step, the inserting (828) step, and guiding (832) the second end of the shaft (101) to the target site (125).

In another example, method (700) may also include the step of coupling (814) a shaver accessory (500) to a working end (550) of the shaft (560) of shaver tool (501). In this example, method (700) may further include the step of second positioning (816) the shaver accessory (500) to, or proximate to, the target site (125). In this example, method (700) may also include the step of actuating (820) blades (580) of the shaver tool (501) to facilitate: removing (822) undesired material or residue from the target site (125), and flowing (824) the undesired material or residue through an opening (508) of the shaver accessory (500) to the first end (554) of the shaft (560) of the shaver tool (501). In one embodiment, at least one of the steps of: coupling (814), second positioning (816), actuating (820), removing (822), and flowing (824), may be performed in method (700) prior to at least one of the: introducing (702), first positioning (704), and depositing (706), steps of method (700).

In yet another example, method (700) may further include the step of identifying (834) tissue to be excised or otherwise removed at, or proximate, the target site (125). In this example, method (700) may include the step of third positioning (836) a tip (614) of a soft tissue removal tool (600) to, or proximate to, the target site (125). In this example, method (700) may also include the step of capturing (838) the tissue to be excised or otherwise removed at, or proximate, the target site (125) using the tip (614). In this example, method (700) may further include the step of withdrawing (840) the soft tissue removal tool (600) from the target site (125) to thereby excise (842) or otherwise remove the tissue from the target site (125). In one embodiment, at least one of the steps of: identifying (834), third positioning (836), capturing (838), withdrawing (840), and excising (842), may be performed in method (700) prior to at least one of the: introducing (702), first positioning (704), and depositing (706), steps of method (700). In another embodiment, at least one of the steps of: identifying (834), third positioning (836), capturing (838), withdrawing (840), and excising (842), may be performed in method (700) prior to at least one of the: coupling (814), second positioning (816), actuating (820), removing (822), and flowing (824), steps of method (700).

In still another example, at least one of the steps of: third positioning (836), capturing (838), and withdrawing (840), may include maneuvering (844) a bent and/or curved distal section (312) of a shaft (602) of the soft tissue removal tool (600) to, or proximal to, the target site (125). In this example, the maneuvering (844) step may include avoiding (846) a portion of a bone positioned proximate to the target site (125) in a joint. In some embodiments, the portion of the bone is, or includes, a head of a femur and the joint is a hip joint, and the bent and/or curved distal section (312) of tool (600) shaft (602) facilitates the maneuvering (844) as described above.

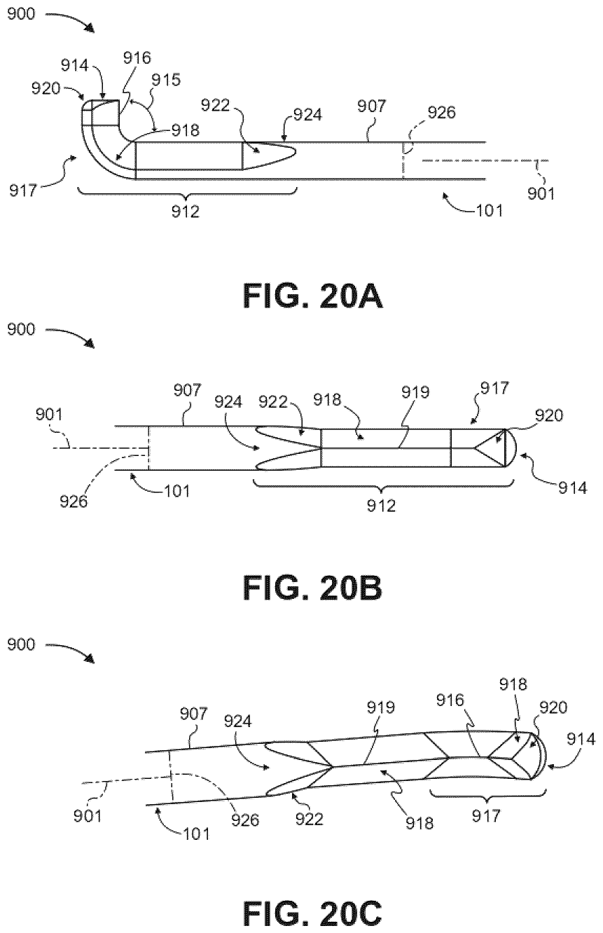

FIGS. 20A-20C depict side, pan and perspective views, respectively of an example soft tissue removal tool (900) having a cutting edge (916) that can be used with the surgical tool kit shown in FIG. 1, to, for example, cut or clear cyst and other soft tissue at or near the target site of a bone abnormality. In one embodiment, soft tissue removal tool (900) may be included in the kit shown and described above with reference to FIG. 1. Soft tissue removal tool (900)—hereinafter referred to more succinctly as tool (900)—includes a rigid shaft (907). In an example, shaft (907) may be formed as a solid (e.g., non-cannulated) piece. In another example, shaft (907) may be formed at least in part as a hollow piece so as to reduce a weight of tool (900). In some embodiments, shaft (907) may be tapered and may have a first section distal to tip (914) having a first outer diameter and a second section proximate to the tip (914) having a second outer diameter that is less than the first outer diameter. In the embodiment illustrated in FIGS. 20A-20C, tool (900) includes a means (926) for detachably coupling an end of shaft (907) distal the tip (914) to another shaft (e.g., shaft (101) as shown and described above with reference to FIG. 1. For instance, such means (926) may include mating screw threads for the detachable coupling, as shown in FIG. 2A.

A first section (912) of tool (900) includes structural features that facilitate cutting or otherwise clearing and/or removing cyst and/or other soft tissue at or near the target site of a bone abnormality. Such features of tool (900) include a cutting edge (916) that may be sharpened into a knife blade. Cutting edge (916) may be formed along, on, or in, all or part of a length of a first section (912) of tool (900). In one embodiment, shaft (907) may be formed of a solid (e.g., non-cannulated) piece of material (e.g., a metal). In another embodiment, shaft (907) may be formed as a hollow construction. In either case, shaft (907) and/or first section (912) including at least a portion of cutting edge (916) may be fabricated or otherwise manufactured by, for example, and without limitation, machining, cutting, a molding process, or combinations thereof. In one example, cutting edge (916) may be formed in first section (912) up to and including at least a portion of tip (914). In another example, cutting edge (916) may be formed in first section (912) but not in at least a portion of tip (914). In some embodiments, as shown in FIGS. 20A-20C, tip (914) may be at least partially rounded, as in at least partially hemispherically, or at least partially half-hemispherically, shaped. First section (912) of tool (900) may include a curved (or bent) section (917) proximate to, and including, tip (914), where an angle (915) orientation of tip (914) relative to an axis (901) of shaft (907) is non-zero. In the illustrated embodiment, tip (914) is oriented at an angle (915) that is at or near normal to axis (901). In other embodiments, not shown in FIGS. 20A-20C, angle (915) may be 0°, greater than 0°, greater than 90°, or greater than 0° and less than or equal to 90°.

In an example, shaft (907) may be longitudinally circular, ellipsoid or oval in cross-section shape. In one example, a cross-sectional shape of shaft (907) is substantially consistent from the end of shaft (907) and/or tool (900) proximate tip (914) to the opposite shaft (907) and/or tool (900) end distal tip (914). In another embodiment, the cross-sectional shape of shaft (907) may vary from the end of shaft (907) and/or tool (900) proximate tip (914) to the opposite shaft (907) and/or tool (900) end distal tip (914). First section (912) of tool (900) may be formed with a pair of cutting edge bevels (918) on one radial side of shaft (907), where cutting edge (916) is formed on a radially outermost juncture of bevels (918). In embodiments of tool (900) having curved section (917) as described above, cutting edge (916) may include a substantially straight (e.g., at or near parallel to axis (901)) section (919). In some embodiments, a portion of an overall length of cutting edge (916) (including arcuate length, where the overall length of edge (916) is greater than a length of first section (912)) occupied by straight section (919) is greater than or equal to 50% of the overall length of cutting edge (916). In other embodiments, not shown in FIGS. 20A-20C, the portion of the overall length of cutting edge (916) occupied by straight section (919) is less than 50%.

Cutting edge (916) may be formed in first section (912) of tool (900) starting from a proximate bevel (920) at, or proximate to, tip (914). Cutting edge (916) may continue distally from tip (914) along at least a portion of first section (912) to a juncture with a flute (924) formed in a portion of first section (912) and/or shaft (907), as shown in FIGS. 20A-20C. A pair of flute bevels (922) may be formed on radially adjacent sides of flute (924), where flute bevels (922) extend away from flute (924) circumferentially from flute (924) on radial sides thereof. The overall length of tool (900) may be selected according to the particular anatomical dimensions of, or other practical considerations for, patients upon which tool (900) may be used.

It is to be understood that the above-described embodiments of the soft tissue removal and other surgical tools, shaver accessory, and/or shaver tool may be interchangeable for use with the kit shown and described above with reference to FIG. 1, for example. Thus, while head (132) is described with reference to a specified structure and including particular features such as aperture (120), as shown in FIG. 1, the various embodiments of soft tissue removal tools, shaver accessory and/or shaver tool, whether they include aperture (120) or not, as the case may be, may be considered as variations of the head (120) shown and described with reference to FIG. 1. As such, embodiments of head (120), along with embodiments of the soft tissue removal tools, shaver accessory, and/or shaver tool may be interchangeable for use, for their respectively described functions, with the kit shown and described above with reference to FIG. 1.

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. The word “or,” in reference to a list of two or more items, covers all of the following interpretations of the word: any of the items in the list, all of the items in the list, and any combination of the items in the list.

The above detailed description of embodiments of the disclosure is not intended to be exhaustive or to limit the teachings to the precise form disclosed above. While specific embodiments of, and examples for, the disclosure are described above for illustrative purposes, various equivalent modifications are possible within the scope of the disclosure, as those skilled in the relevant art will recognize. For example, while processes or blocks are presented in a given order, alternative embodiments may perform routines having steps, or employ systems having blocks, in a different order, and some processes or blocks may be deleted, moved, added, subdivided, combined, and/or modified to provide alternative or subcombinations. Each of these processes or blocks may be implemented in a variety of different ways. Also, while processes or blocks are, at times, shown as being performed in a series, these processes or blocks may instead be performed in parallel, or may be performed at different times. Further, any specific numbers noted herein are only examples: alternative implementations may employ differing values or ranges.

The teachings of the disclosure provided herein can be applied to other systems, not necessarily the system described above. The elements and acts of the various embodiments described above can be combined to provide further embodiments.

Any patents and applications and other references noted herein, including any that may be listed in accompanying filing papers, are incorporated herein by reference. As to aspects of the disclosure can be modified, if necessary, to employ the systems, functions, and concepts of the various references described above to provide yet further embodiments of the disclosure.

These and other changes can be made to the disclosure in light of the above Detailed Description. While the above description describes certain embodiments of the disclosure, and describes the best mode contemplated, no matter how detailed the above appears in text, the teachings can be practiced in many ways. Details of the system may vary considerably in its implementation details, while still being encompassed by the subject matter disclosed herein. As noted above, particular terminology used when describing certain features or aspects of the disclosure should not be taken to imply that the terminology is being redefined herein to be restricted to any specific characteristics, features, or aspects of the disclosure with which that terminology is associated. In general, the terms used in the following claims should not be construed to limit the disclosure to the specific embodiments disclosed in the specification, unless the above Detailed Description section explicitly defines such terms. Accordingly, the actual scope of the disclosure encompasses not only the disclosed embodiments, but also all equivalent ways of practicing or implementing the disclosure under the claims.

While certain aspects of the disclosure are presented below in certain claim forms, the inventors contemplate the various aspects of the disclosure in any number of claim forms. For example, while only one aspect of the disclosure is recited as a means-plus-function claim under 35 U.S.C. § 112(f), other aspects may likewise be embodied as a means-plus-function claim, or in other forms, such as being embodied in a computer-readable medium. (Any claims intended to be treated under 35 U.S.C. § 112(f) will begin with the words “means for”.) Accordingly, the applicant reserves the right to add additional claims after filing the application to pursue such additional claim forms for other aspects of the disclosure.

The detailed description provided herein may be applied to other systems, not necessarily only the system described above. The elements and acts of the various examples described above can be combined to provide further implementations of the invention. Some alternative implementations of the invention may include not only additional elements to those implementations noted above, but also may include fewer elements. These and other changes can be made to the invention in light of the above Detailed Description. While the above description defines certain examples of the invention, and describes the best mode contemplated, no matter how detailed the above appears in text, the invention can be practiced in many ways. Details of the system may vary considerably in its specific implementation, while still being encompassed by the invention disclosed herein. As noted above, particular terminology used when describing certain features or aspects of the invention should not be taken to imply that the terminology is being redefined herein to be restricted to any specific characteristics, features, or aspects of the invention with which that terminology is associated. In general, the terms used in the following claims should not be construed to limit the invention to the specific examples disclosed in the specification, unless the above Detailed Description section explicitly defines such terms. Accordingly, the actual scope of the invention encompasses not only the disclosed examples, but also all equivalent ways of practicing or implementing the invention.

What is claimed is:

1. A surgical tool comprising:
a shaft;
a tip section located at an end of the shaft and comprising a blunt surface defining a distal end of the surgical tool;
a cutting section extending from the tip section and along a first portion of the shaft, the cutting section defining a cutting edge having a curved portion adjacent the tip section and a straight portion extending from the curved portion; and
a base section extending along a second portion of the shaft and configured to attach to a handle.

2. The surgical tool of claim 1, wherein the curved portion of the cutting edge is positioned on an opposing side of the shaft with respect to the blunt surface.

3. The surgical tool of claim 2, wherein the curved portion of the cutting edge is oriented at a non-zero angle with respect to a longitudinal axis of the shaft.

4. The surgical tool of claim 1, wherein:
the shaft is cylindrical; and
the cutting edge is formed by removing material from the shaft.

5. The surgical tool of claim 4, wherein the cutting section comprises one or more bevels extending between the cutting edge and a cylindrical portion of the shaft.

6. The surgical tool of claim 1, wherein the base section is configured to be removably attached to the handle.

7. The surgical tool of claim 1, wherein the handle is fixed to the shaft.

8. The surgical tool of claim 1, wherein the blunt surface is at least partially rounded.

9. A surgical cutting tool comprising:
a shaft defining:
a tip section located at a first end of the shaft and comprising a blunt surface defining a distal end of the surgical cutting tool;
a cutting section extending from the tip section and comprising a cutting edge having a curved portion adjacent the tip section and a straight portion extending from the curved portion; and
a base section located at a second end of the shaft, opposite the first end; and
a handle coupled to the base section.

10. The surgical cutting tool of claim 9, wherein:
the shaft is cylindrical; and
the tip section and the cutting section are formed in the shaft.

11. The surgical cutting tool of claim 10, wherein:
the shaft is formed from a metal material; and
the cutting edge is formed by removing a portion of the metal material.

12. The surgical cutting tool of claim 9, wherein the curved portion of the cutting edge is formed on an opposing side of the shaft to the blunt surface.

13. The surgical cutting tool of claim 12, wherein the curved portion of the cutting edge is oriented at a non-zero angle with respect to a longitudinal axis of the shaft.

14. The surgical cutting tool of claim 13, wherein the curved portion of the cutting edge is at an angle greater than ninety degrees with respect to the straight portion of the cutting edge.

15. The surgical cutting tool of claim 13, wherein the straight portion of the cutting edge is oriented substantially parallel to the longitudinal axis of the shaft.

16. The surgical cutting tool of claim 9, wherein:
the tip section comprises a cylindrical portion; and
the cutting section comprises one or more bevels extending between the cutting edge and the cylindrical portion.

17. The surgical cutting tool of claim 9, wherein the blunt surface is at least partially hemispherical.

18. The surgical cutting tool of claim 9, wherein the blunt surface extends to the curved portion of the cutting edge.

19. The surgical cutting tool of claim 9, wherein a length of the straight portion of the cutting edge is greater than a length of the curved portion of the cutting edge.

20. The surgical cutting tool of claim 19, wherein the shaft defines one or more bevels between a cylindrical portion of the shaft and the cutting edge.
